(19) United States
(12) Patent Application Publication
(10) Pub. No.: US 2003/0096237 A1
(43) Pub. Date: May 22, 2003

(54) Determination of the ability of patients to respond to a tumor treatment

(76) Inventor: Ulrich Certa, Allschwil (CH)

Correspondence Address: HOFFMANN-LA ROCHE INC., PATENT LAW DEPARTMENT, 340 KINGSLAND STREET, NUTLEY, NJ, 07110

(21) Appl. No.: 09/931,733

(22) Filed: Aug. 17, 2001

(30) Foreign Application Priority Data
Aug. 28, 2000 (EP) 00118603.0

(51) Int. Cl. C12Q 1/68; A61K 38/21

(52) U.S. Cl. 435/6; 424/85.7

(57) ABSTRACT

The present invention relates to a method for screening patients to determine their ability to respond to a tumor treatment and also to a diagnostic test for carrying out the said method.

### FIELD OF THE INVENTION

The present invention relates to a method for screening patients to determine their ability to respond to a tumor treatment. It concerns also a diagnostic test for carrying out the said method.

### BACKGROUND OF THE INVENTION

Treatment capable of opposing the development of tumors have a lot of interest in the therapeutically research. Since not all tumors are the same, only some of the patients respond to a particular tumor treatment.

Furthermore, in subgroups of patients, specific activities of tumor treatments are accompanied by toxic effects for health, including, for example, gastrointestinal disorders, hypo- or hypertension, tachycardia, fatigue/asthenia or headache. In other subgroups of patients, no obvious beneficial effects are observed.

Finally, tumor cells change over time and may eventually become resistant to a specific tumor treatment.

Carcinogenesis, tumor progression and metastasis result from an imbalanced transcriptional program, inappropriate post-translational modifications and deregulated epigenetic modifications (Schwirzke, M. et al., Anticancer Res 19 (1999) 1801-1814; Pardee, A. B., Advances in Cancer Res 65 (1994) 213-227; Ponta, H., Biochim Biophys Acta 1198 (1994) 1-10). Changes of the transcriptional program are due to oncogenes and tumor suppressor genes, fusion proteins created by cytogenetic alterations, altered expression of genes due to unscheduled methylation by DNA methyltransferases and chromatin modifying enzymes such as histone acetyltransferases and histone deacetylases (Lin, R. J. et al., Trends Genet 15 (1999) 179-184; Stunnenberg, H. G. et al., Biochem Biophys Acta 1423 (1999) F15-F33).

One major difficulty is that, at present, it is only rarely possible to predict whether a particular patient's tumor will respond to a specific tumor treatment. It is also difficult to predict how tumor cells change over time and if they become resistant to a specific tumor treatment.

For identification of tumor-related candidate genes, transcriptional profiling of cellular systems such as metastasizing versus non-metastasizing cell lines and tumor specimen corresponding to different stages of progression is the first step for achievement of this goal (Schiemann, S. et al., Anticancer Research 17 (1997) 13-20; Schwirzke, M. et al., Anticancer Research 18 (1998) 1409-1422; Schiemann, S. et al., Clin Exp Metastasis 16 (1998) 129-139). Further steps involve analysis of prevalence of the identified alteration in different tumors, in-vitro modulation of the gene under consideration by overexpression and downregulation making use of antisense RNA or ribozymes in stable transfectants and assessing the consequences in relevant in-vitro systems. The advent of nude mouse systems, including subcutaneous xenograft systems and orthotopic implantation in which the natural tropism of metastasis of the tumor under investigation is maintained, has paved the way for assessment of the functional role of candidate genes in vivo (Fidler, I. J., Cancer Metastasis Rev 50 (1986) 29-49).

Clinical, immunological or molecular features enabling a targeted selection of patients likely to take advantage of tumor treatments have not been identified so far.

Clearly, the possibility to develop criteria predicting the potential effectiveness of tumor treatments would be of high clinical relevance since it would spare unnecessary toxicity to non responders and it would contribute to the identification of specific responder patients' subgroups.

### SUMMARY OF THE INVENTION

The present invention provides a method for screening patients to determine their ability to respond to a tumor treatment, said method comprising measuring by said patients the expression level of one or more genes responsive for said treatment and comparing the result of measurement to a reference sample. The present invention also concerns a diagnostic tests for carrying out said method.

In another aspect, the invention provides a method for determining in a patient sample originating from a tumor, the presence or absence of a gene predicative for treatment of the tumor with IFN-α or a pegylated IFN-α conjugate. The method comprises obtaining from a patient having a tumor, a sample containing cells originating from the tumor; and detecting in the patient sample the expression of the gene. The presence of the gene is predicative of the patient having an ability to respond to the treatment of the tumor using IFN-α or a pegylated IFN-α conjugate.

Preferably, the tumor is ovary cancer, prostate cancer, breast cancer, colon cancer, liver cancer, stomach cancer or lung cancer. Preferably, the patient sample is prepared from blood, urine, serum, lymph node, bone marrow, cell extracts or tissue extracts. Preferably, the sample contains melanoma cells.

Preferably, the pegylated-IFN-α conjugate has the formula:

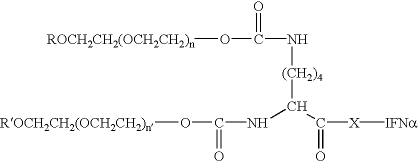

wherein R and R′ are independently lower alkyl; X is NH or O; n and n′ are integers having a sum of from 600 to 1500; and the average molecular weight of the polyethylene glycol in said conjugate is from about 26,000 Daltons to about 66,000 Daltons. More preferably, the pegylated-IFN-α conjugate has the formula:

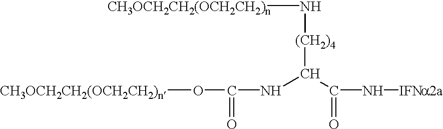

wherein n and n′ are independently 420 or 520.

Preferably, the gene is at least one gene selected from the group consisting of S75415, M32053, X16665, and D00597.

### BRIEF DESCRIPTION OF THE DRAWINGS

FIG. 1 shows expression of genes encoding tumor associated antigens and IFN-α receptor in melanoma cell lines. Panel A shows expression of genes encoding tumor associated antigens. Panel B shows IFN-α receptor gene expression is detected in IFN-α sensitive and resistant melanoma cell lines by 25 cycles RT-PCR.

FIG. 2 shows genes preferentially expressed in IFN-α sensitive or resistant melanoma cell lines. Panel A and Panel B show combined data sets for oligonucleotide array expression data collected from the sensitive (CNCM I-2544 (A375), CNCM I-2546 (ME15), CNCM I-2547 (ME51) and CNCN I-2548 (ME59)) or resistant cell lines (CNCM I-2545 (D10) and CNCM I-2549 (ME67)).

### DETAILED DESCRIPTION OF THE INVENTION

The expression “tumor treatments” as used herein includes all biological or chemical antitumor drugs.

The expression “reference sample” as used herein concerns a reference of gene expression level characteristic of normal cells or of normal tissue extracts.

The term “IFN-α” as used herein includes IFN-αs derived from any natural material (e.g., leukocytes, fibroblasts, lymphocytes) or material derived therefrom (e.g. cell lines), or those prepared with recombinant DNA technology. Details of the cloning of IFN-α and the direct expression thereof, especially in E. coli, have been the subject of many publications. The preparation of recombinant IFN-αs is known, for example from Goeddel et al. (1980) Nature 284, 316-320 and (1981), Nature 290, 20-26, and European Patents Nos. 32134, 43980 and 211148. There are many types of IFN-α such as IFN-αI, IFN-α2; and further their subtypes including but not limited to IFN-α2A, IFN-α2B, IFN-α2C and IFN-αII (also designated IFN-αII or w-IFN). In the present invention, the use of IFN-α2A is preferred. The manufacture of IFN-α2A is described in European Patents Nos. 43980 and 211148.

The method of the present invention for screening patients to determine their ability to respond to a tumor treatment comprises measuring the expression level of at least one of the genes predictive for said treatment in patient samples and comparing the result of the measurement with the result obtained with a reference sample.

The present invention is useful for screening patients suffering from tumor. Exemplary tumors include ovaries cancer, prostate cancer, breast cancer, colon cancer, liver cancer, stomach cancer or lung cancer. Of particular interest is melanoma cancer.

In the present invention, the tumor treatment may include tumor drugs like cytokine, for example.

It may include an active ingredient IFN-α or pegylated IFN-α conjugate in a therapeutically effective amount to decrease the severity of the viral infection.

The IFN-α used in this invention may be conjugated to a polymer such as a polyalkylene glycol (substituted or unsubstituted), for example polyethylene glycol, to form PEG-IFN-α. Conjugation may be accomplished by means of various linkers known in the art, in particularly by linkers such as those disclosed in European Patent Applications, Publication Nos. 0510356 and 593868 and European Patent Application No. 97108261.5. The molecular weight of the polymer, which is preferably polyethylene glycol, may range from 300 to 30.000 Dalton, and one or more, preferably one to three, polymers may be conjugated to the IFN-α.

Most preferably, the reagents attach to primary amino groups on for example lysine or to the N-terminus of the IFN-α. The reagents can also attach to a hydroxyl on for example serine. One or more, preferably one to three, PEGs may be conjugated to the IFN-α.

A most preferred reagent is of the formula

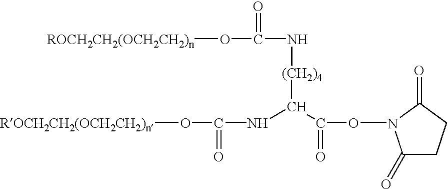

in which a total of 2 monomethoxy PEG (m-PEG) chains is linked to lysine, one at the α and ε amino groups via carbamate (urethane) bonds and the lysine carboxyl group is activated to a succinimidyl ester. This reagent may be obtained by conventional means, according to known procedures (Monfardini et al., “A branched monomethoxypoly(ethylene glycol) for protein modification”, Bioconjugate Chem. 6:62, 1995) applicable to a reagent with R being lower alkyl and having a desired n. This reagent may be obtained from Shearwater Polymers, Inc. (Huntsville, Ala.). The preferred average molecular weight of the PEG is about 20,000 Daltons, providing a total PEG mass of about 40,000 Daltons in PEG2-NHS (other molecular weights may be obtained by varying n for the PEG-alcohol starting materials for the reagent of the above formula, by conventional means.

A preferred pegylated-IFN-α conjugate has the formula:

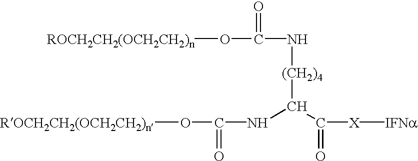

wherein R and R′ are independently lower alkyl; X is NH or O; n and n′ are integers having a sum of from 600 to 1500; and the average molecular weight of the polyethylene glycol in said conjugate is from about 26,000 Daltons to about 66,000 Daltons.

Most preferred is the pegylated interferon-α is of the formula

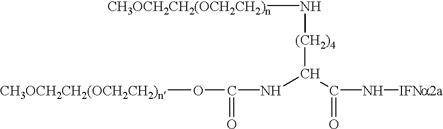

wherein n and n′ are independently 420 or 520. This pegylated IFN-α conjugate is known, for example in European Patent Application EP 809996, incorporated herein by reference.

The IFN-α used in the present invention may be associated with other active pharmaceutical compounds, like mycophenolate moftil, ribavirin or amantadine.

Measurement of the expression level of genes in the present invention can be carried out in a variety of ways:

in situ hybridization with fixed whole cells, with fixed tissue samples,

Southern hybridization (DNA detection) (Sambrook et al., Molecular Cloning, vol. 2, 9.31-9.57),

Northern hybridization (RNA detection) (Sambrook et al., Molecular Cloning, vol. 1, 7.37 & 7.39),

serum analysis (e.g., cell type analysis of cells in the serum by slot-blot analysis),

after amplification (e.g., PCR technique) (Sambrook et al., Molecular Cloning, vol. 2, 14.2-14.4).

Gene expression may be measured directly by RNA analysis such as Northern blot or Dot blot techniques. Such blotting techniques require the use of nucleic acid probes, usually radiolabelled, specific to at least one of the genes predictive for said treatment. Probes may be prepared synthetically based on the known nucleotide sequences of the predictive genes of reference sample. For Northern blotting RNA is obtained from tissue extracts by conventional methods. The RNA is then denatured and separated on an agarose gel by elecrophoresis followed by transfer to a nylon or a cellulose nitrate filter by blotting and fixation by baking. The filter is then exposed to a single labeled complementary probe and mRNA of interest is detected, usually, by autoradiography. Dot blotting is similar except that the mRNA is not electrophoresed before immobilization. In situ hybridization may also be used to measure gene expression by the level of mRNA.

Alternatively, gene expression may be measured by the level of gene product by Western blotting and by immunohistochemical staining, for example. According to Western blotting measurement, predictive proteins of the reference sample and the sample to be tested or peptides of said proteins are separated by SDS polyacrylamide gel electrophoresis and transfered on to nitrocellulose membrane. After washing, the nitrocellulose membrane is then incubated with antibodies labeled with radioactive or fluorescence labels. According to immunohistochemical staining antibodies may be either monoclonal or polyclonal and may be prepared against a synthetic peptide based on the reported DNA of at least one predictive gene of a reference sample. Those synthetic peptides may then be used as immunogen in preparing antibodies by well-known techniques. Immunogen may be also directly prepared from the native product of at least one of the predictive genes for tumor treatment and/or portions thereof.

In the present invention, for determining the ability of patients to respond to a tumor treatment, patient samples are prepared depending on the nature of the cancer. Patient samples including blood, urine, serum, lymph node, bone marrow, cell extracts or tissue extracts may be used, for example.

The lymph node may be fresh samples or frozen, preferably snap-frozen in liquid nitrogen, and stored at about −80° C. Blood and bone marrow samples, upon collection, are stored and their cells lysed using preferably a guanidine hydrochloride-based solution with detergent. Additional steps to enrich specific cell fractions in blood, such as Ficoll-gradient separation, can also be used prior to cell lysis.

If samples such as blood or bone marrow are used, it is preferred to perform an enrichment of epithelial cells or lymphocytes prior to mRNA extraction. Such an enrichment can be done by the use of epithelial specific binding such as immuno beads or high gradient and magnetic cell sorting (MACS) (Hardingham, I. E., et al., Int. J. Cancer 20 (2000) 8-13; Martin, V. M., et al., Exp. Hematology 26 (1998) 252-264).

The diagnostic test in the present invention comprises contacting a matrix with probes like nucleic acid or protein probes with a liquid phase containing antibodies or nucleic acid probes and detecting gene transcription or product of one of the genes predictive for tumor treatment.

For detecting the presence of the product of one gene predictive for said tumor treatment, the antibodies in the liquid phase may be bound to a marker. Particularly, the enzyme alkaline phosphatase is used as marker.

The present invention also relates to a kit for determining the ability of patients to respond to a tumor treatment, said kit comprising a container with a matrix with probes. Preferably, the probes on the matrix are nucleic acids.

The therapeutic efficacy of tumor treatment may be related to direct effects on the cells.

It would be useful to have a method for screening the availability of cells to be sensitive or resistant to tumor treatment, said method comprising the identification of gene expression profiles specific of said treatment. Cells may be one or more cell lines, especially tumor cell lines and more particularly melanoma primary cell lines.

Another object of the present invention concerns an immunological marker enabling the selection of cells responding to a tumor treatment. This immunological marker is an antibody specific for a product of one or more of the genes predictive for said tumor treatment.

Finally the present invention concerns a diagnostic test for determining whether or not a cancer cell-containing test sample originating from or containing human cells has potential for tumor development, tumor progression or metastasis with a specific tumor treatment, wherein the test sample and a second sample originating from non-tumor cells obtained from the same individual or a different individual of the same species, which test comprises the following steps:

incubating each respective sample under stringent hybridization conditions with a nucleic acid probe which is selected from the group consisting of:

a nucleic acid sequence of at least one of the genes predictive for said treatment;

a nucleic acid sequence which is complementary to any nucleic acid sequence of (i);

a nucleic acid sequence which hybridizes under stringent conditions with the sequence of (i); and

a nucleic acid sequence which hybridizes under stringent conditions with the sequence of (ii); and

determining the approximate amount of hybridization of each respective sample with said probe, and

comparing the approximate amount of hybridization of the test sample to an approximate amount of hybridization of said second sample to identify whether or not the test sample contains a greater amount of the specific nucleic acid or mixture of nucleic acids than does said second sample.

Typically, the approximate amount of hybridization is determined qualitatively, for example, by a sight inspection upon detecting hybridization. For example, if a gel is used to resolve labelled nucleic acid which hybridizes to target nucleic acid in the sample, the resulting band can be inspected visually. One can compare the approximate amount of hybridization in the test sample to the approximate amount of hybridization in non-tumor cells. Such non-tumor cells are, e.g., epithelial cells or peripheral blood cells.

The present invention will be better understood on the basis of the following examples, offered by way of illustration and not by way of limitation.

The cell lines used in the present invention can be any human cell lines. Especially, preferred are the responder cell lines CNCM I-2544, CNCM I-2546, CNCM I-2547 and CNCM I-2548 and the non responder cell lines CNCM I-2545 and CNCM I-2549 isolated from human primary melanoma. Cultures of these cell lines were deposited and registered under the Budapest Treaty (Rule 6.1) by the Culture Collection “Collection Nationale de Cultures de Microorganismes” on Aug. 17, 2000.

### EXAMPLES

The examples below are in connection with the following figures:

FIG. 1. Expression of genes encoding tumor associated antigens and IFN-α receptor in melanoma cell lines.

Panel A: Expression of genes encoding tumor associated antigens.

The cell lines CNCM I-2544 (375), CNCM I-2545 (D10), CNCM I-2546 (15), CNCM I-2547 (51), CNCM I-2548 (59) and CNCM I-2549 (67) were cultured for 48 hours in the presence (+) or absence (−) of 100 U/ml IFN-α. The expression patterns of genes encoding tyrosinase, tyrosinase related protein-2 , pmel-17 and mart-1, HLA restricted, tumor associated antigens are reported. Grey bars refer to IFN-α sensitive cell lines and black bars refer to IFN-α resistant lines (see Table 1). Data are presented as average difference of signal intensity between match and mismatch probesets.

Panel B: IFN-α receptor gene expression is detected in IFN-α a sensitive and resistant melanoma cell lines by 25 cycles RT-PCR.

FIG. 2. Genes preferentially expressed in IFN-α sensitive or resistant melanoma cell lines.

Oligonucleotide array expression data were collected from untreated cells. Data from the sensitive (CNCM I-2544 (A375), CNCM I-2546 (ME15), CNCM I-2547 (ME51) and CNCN I-2548 (ME59)) or resistant cell lines (CNCM I-2545 (D10) and CNCM I-2549 (ME67)) were combined into two data sets (panel A and panel B). Average values for individual genes were then filtered to identify genes upregulated at least three fold in either group. Data are presented as average difference of signal intensity between match and mismatch probesets.

Identification of IFN-α inducible genes in melanoma cell lines

Cell Lines and Culture Conditions

Melanoma cell lines were screened for their sensitivity to proliferation inhibition and HLA class I induction. The study was done of six cell lines (CNCM I-2544, CNCM I-2545, CNCM I-2546, CNCM I-2547, CNCM I-2548 and CNCM I-2549).

All these cell lines were cultured in RPMI medium supplemented with 10% FCS, glutamine (2 mM), sodium pyruvate (1 mM), non-essential amino acids and HEPES buffer (10 mM) (all from GIBCO Life Sciences, Paisley, UK). When confluent, the cells were passaged by trypsinization.

Oligonucleotide Array Analysis

Cultured melanoma cells were harvested by scraping and total cellular RNA was extracted. 10 μg from each sample were used directly as templates for cDNA synthesis using a commercial kit (Roche Molecular Biochemicals, Rotkreuz, Switzerland). The T7 promoter sequence incorporated into the cDNA synthesis primer allowed template amplification and biotin labeling by in vitro transcription using a commercial kit (Affymetrix, Santa Clara, Calif.). After alkaline heat fragmentation cDNA was hybridised to the array (Affymetrix, Santa Clara, Calif.) and all subsequent steps were performed following standard procedures as supplied with the arrays. Raw data were collected with a confocal laser scanner (Hewlett Packard, Palo Alto, Calif.) using GeneChip software v3.1 (Affymetrix, Santa Clara, Calif.).

Data Analysis

Raw data were normalized based on the total chip signal obtained upon hybridisation of ME15 cell line cDNA. This array was selected because: i) the 5′/3′ intensity ratio of control genes was smaller than 3; ii)>25% of the genes are called “present” by Genechip; iii) the image appearance is homogeneous with low background and no sign of mechanical chip damage.

The normalized average difference (nAD) between the signals of the perfect and of the mismatch probesets for each gene was used as the expression level of a given gene. At least one nAD value in a pairwise comparison of data has to exceed 50 to be included. Based on nAD, change factors related to IFN-α exposure were also calculated. Negative nAD values were set to 20 in order to avoid the calculation of artificially high modifications. In order to exclude artefacts, only genes with robust change factors, greater than 2-fold, were included in the analysis. By applying these criteria, about 60-80 genes were found to be differentially expressed depending on the cell line analyzed. Array to array variations did not exceed 2% based on the hybridization of one sample to 5 arrays from the same batch in a pilot study. Genes were clustered according to their mode of regulation.

Identification of IFN-α Sensitive and Resistant Melanoma Cell Lines

A number of established melanoma cell lines were assayed for their sensitivity to IFN-α by testing the capacity of IFN-α to inhibit their proliferation and to increase their surface expression of HLA class I determinants.

Two cell lines (CNCM I-2545 and CNCM I-2549) were found to be resistant to the antiproliferative effects of IFN-α.

Proliferation of CNCM I-2547 and CNCM I-2548 could be at least 50% inhibited by IFN-α concentrations as low as 10 U/ml, whereas CNCM I-2544 and CNCM I-2546 required a ten times higher dose for the elicitations of similar effects (Table 1).

The upregulation of HLA class I expression by IFN-α closely matched its antiproliferative effects. In no case a dissociation of the two activities could be observed (Table 1).

Table 1. Identification of melanoma ceU lines sensitive or resistant to the inhibition of proliferation and to the HLA class I overexpression induced by IFN-α.

| | proliferation inhibition | |
|---|---|---|
| Cell line | (IC50) (a) | HLA class I induction (b) |
| CNCMI-2544 | +(100 U/ml) | (442 vs. 305) |
| CNCMI-2545 | − | (184 vs. 196) |
| CNCMI-2546 | +(100 U/ml) | (521 vs. 257) |
| CNCMI-2547 | +(10 U/ml) | (980 vs. 135) |
| CNCMI-2548 | +(10 U/ml) | (559 vs. 380) |
| CNCMI-2549 | − | (175 vs. 203) |

Melanoma cell lines were cultured in the presence of IFN-α concentrations ranging between 1 and 1000 U/ml. 3H-Thymidine incorporation was measured daily over a five days culture period following an 18 hour pulsing time. IC50 is the IFN-α concentration inducing at least a 50% inhibition of a maximal proliferation activity detectable in individual experiments.
Melanoma cells were stained with HLA class I specific monomorphic mAbs following a two days culture in the presence (digits on the left) or absence (digits on the right) of IFN-α (100 U/ml) and tested by flow-cytometry. Data are expressed as mean fluorescence intensity of labelled cell lines.

Detection of Genes Encoding Tumor Associated Antigens in Melanoma Cell Lines

Total cellular RNA was extracted from the sensitive and resistant melanoma cell lines identified above, reverse transcribed and processed for hybridization to an oligonucleotide array (Hu6800FL, PN 900183) containing probe sets derived from 7000 full-length human genes. Expression levels for each gene were calculated as normalized average difference (nAD, see materials and methods).

Datasets for genes encoding MART-1/Melan-A, pmel-17 (gp100), TRP-2 and tyrosinase tumor associated antigens (TAA) were first analyzed. These four genes were found to be expressed in CNCM I-2546 and CNCM I-2545 cell lines, whereas virtually no expression was detectable in CNCM I-2544, CNCM I-2546, CNCM I-2547 and CNCN I-2548 cell lines (FIG. 1). Functional tests confirmed these findings. Indeed, CNCM I-2545, HLA-A2.1 positive melanoma cells were effectively killed by HLA-A2.1 restricted CTL lines recognizing epitopes derived from MART-1/Melan-A, pmel-17/gp100, tyrosinase or TRP-2 proteins. In contrast, CNCM I-2548 HLA-A2.1 positive cells, that do not express the genes under investigation failed to be killed by the specific CTL (FIG. 1). Remarkably, IFN-α treatment does not appear to influence the expression of the genes encoding these TAA.

Thus, the application of the microarray technology to the cellular system under investigation is validated by results concurrently obtained at functional and gene expression levels.

IFN-α Receptor Gene Expression in Melanoma Cell Lines

A differential sensitivity to the effects of IFN-α is related to a differential expression of the specific receptor. Indeed, transcripts from the IFN-α receptor gene (IFNAR2) were detected at low levels, nAD≦60, (Table 2, gene cluster 5) upon microarray hybridization of the cDNA from the cell lines under investigation.

IFN-α receptor gene expression was evaluated by using a more sensitive RT-PCR assay (FIG. 2B).

Although to different extents, unrelated to the level of responsiveness to IFN-α, specific transcripts were detectable in all lines.

Detection of Potential Marker Genes for IFN-α Responsiveness

The genetic profile of melanoma cell lines was classified according to their sensitivity or resistance to critical direct effects of IFN-α, namely the inhibition of proliferation and the upregulation of HLA class I expression.

The availability of large mRNA expression data sets from six human melanoma cell lines well mentioned for their responsiveness to IFN-α raised the possibility of identifying genes preferentially expressed in sensitive or resistant lines in the absence of cytokine treatment. Microarray data of all genes from responder (CNCM I-2544, CNCM I-2546, CNCM I-2547 and CNCN I-2548) and non responder (CNCM I-2545 and CNCM I-2549) cell lines were combined resulting in two averaged data sets. These data were then screened for genes upregulated more than three fold in either group. The average data were then dissociated in order to obtain the individual expression levels in each cell line. A gene was considered positive or predictive when the deviation from the mean was lower than 30% of the average value of the nAD.

This analysis resulted in the identification of a group of four genes preferentially expressed in IFN-α sensitive cell lines (FIG. 2, panel A). Two of them, IFI16 and RCC1 encode nuclear proteins endowed with mitotic regulation and transcriptional activation capacities, respectively. A third is the hox2 homeobox gene, whereas the fourth, h19 gene, encodes an untranslated RNA, involved in the DNA methylation and genetic imprinting processes. Notably, however, one of the IFN-α sensitive cell lines, ME51, does not express RCC1.

On the other hand, two genes encoding likely components of signal transduction pathways, SHB and PKC-ζ, appeared to be preferentially expressed in IFN-α resistant D10 and ME67 cell lines (FIG. 2, panel B).

A pattern of genes preferentially expressed according to typical profiles in sensitive and resistant cells clearly emerged. Genes involved in the regulation of cell proliferation, such as IFI16, h19 and RCC1, but also hox2, were found to be preferentially expressed in sensitive cell lines. Intriguingly, genes encoding SHB and PKC-ζ proteins, known components of defined signal transduction pathways appeared to be preferentially expressed in IFN-α resistant cells. These puzzling data suggest that IFN-α resistance could result from a series of active events as opposed to a merely defective activation.

Induction of Gene Expression by IFN-α in Sensitive and Resistant Cell Lines

Patterns of genes expressed in IFN-α sensitive and resistant melanoma cell lines were investigated upon 48 hour culture in the presence of IFN-α. CNCM I-2546 and CNCM I-2545 were studied in detail. Analysis was focused on genes which were at least 3-fold up or down regulated as compared to untreated cells and displayed nAD values of at least 50 in one of the four experiments.

Cluster 1 (Table 2) includes genes only inducible in the sensitive CNCM I-2546 cell line. As expected expression of these genes was not significantly affected by the treatment in IFN-α resistant CNCM I-2545 cells. This set of genes includes HLA class I genes, 2-5A synthetase, TAP-1, genes encoding a number of interferon-inducible proteins, but also p27 cyclin-dependent kinase inhibitor and ROX protein. A single gene, encoding amplaxin or ems-1 and derived from the locus 11q13 frequently amplified in tumor cells, appeared to be induced by IFN-α in both lines (cluster 2).

Cluster 3 genes, including ip-30, a known IFN-γ inducible gene, and dss 1 were induced in the CNCM I-2545 resistant cell line but their expression was not significantly altered in CNCM I-2546 sensitive cells. Cluster 4 includes additional genes inducible by IFN-α in CNCM I-2546 which are, in contrast to cluster 1, downregulated in IFN-α resistant CNCM I-2545 cells. Interestingly, the transcription factor ISGF-3, of relevance for IFN-α signalling, belongs to this cluster that includes other IFN related genes. Cluster 5 comprises genes downregulated by IFN-α treatment in resistant CNCM I-2545 cells, but virtually unaffected in sensitive CNCM I-2546 cells. Interestingly, this cluster comprises the gene encoding IFN-α receptor. Cluster 6 includes two genes (irf-2 and interferon-induced cellular resistance mediator) whose expression, basically undetectable in the CNCM I-2545 resistant cell line, is downregulated in CNCM I-2546 IFN-α sensitive cells. Interestingly, the IRF-2 gene product is known to bind to the promoter region of IFN type I inducible genes and to prevent transcription. Downregulation could thus promote the activation of IFN inducible genes.

In Table 2 below, CNCM I-2546 and CNCM I-2545 cultured for 48 hours in the absence or in the presence of 100 U/ml IFN-α were used to assay cytokine modulated gene expression as matched with data obtained in fibrosarcoma cells. This analysis yielded six clusters of genes. Cluster 1 contains genes only induced in the IFN-α sensitive CNCM I-2546. Cluster 2 includes amplaxin, upregulated in both lines and cluster 3 comprises genes only induced in the CNCM I-2545 resistant line. Cluster 4 refers to genes downregulated in CNCM I-2545 and induced in CNCM I-2546. Cluster 5 includes genes downregulated in CNCM I-2545 and cluster 6 refers to genes downregulated in CNCM I-2546. Data are presented as average difference of signal intensity between match and mismatch probesets.

TABLE 2
IFN-α modulated
genes

| | | CNCM I-2545 | CNCM I-2545 | CNCM I-2546 | CNCM I-2546 | CNCM I-2545 | CNCM I-2546 | |
|---|---|---|---|---|---|---|---|---|
| | | (−) | (+) | (−) | (+) | CF | CF | Cluster |
| Cluster 1 | | | | | | | | |
| U51127 | interferon regulatory | 262 | 91 | 3 | 116 | −1.88 | 4.8 | ˜u |
| | factor 5 | | | | | | | |
| X07730 | prostate specific | 234 | 85 | −64 | 132 | −1.75 | 5.6 | ˜u |
| | antigen | | | | | | | |
| X57522 | ring4 cdna | 143 | 49 | −49 | 97 | −1.92 | 3.85 | ˜u |
| Z56281 | interferon regulatory | 93 | 31 | 12 | 136 | −2 | 5.8 | ˜u |
| | factor 3 | | | | | | | |
| J04164 | interferon-inducible | −447 | −97 | −89 | 2066 | 0 | 102.3 | ˜u |
| | protein 27-sep | | | | | | | |
| U50648 | interferon-inducible | 106 | 46 | −70 | 71 | −1.3 | 2.55 | ˜u |
| | rna-dependent | | | | | | | |
| | protein kinase (pkr) | | | | | | | |
| X67325 | p27 | −460 | −86 | −211 | 410 | 0 | 19.5 | ˜u |
| HG658 | major | 1183 | 455 | 156 | 1692 | −1.6 | 9.85 | ˜u |
| | histocompatibility | | | | | | | |
| | complex, class 1 c | | | | | | | |
| M13755 | interferon-induced | −163 | 31 | −181 | 716 | 0.55 | 34.8 | ˜u |
| | 17-kda/15-kda | | | | | | | |
| | protein | | | | | | | |
| D28137 | bst-2, | 37 | 88 | 28 | 305 | 1.38 | 9.89 | ˜u |
| M19650 | 2′,3′-cyclic | 137 | 99 | 55 | 269 | −0.38 | 3.89 | ˜u |
| | nucleotide 3prime- | | | | | | | |
| | phosphodiesterase | | | | | | | |
| X96401 | rox protein | 229 | 83 | 34 | 143 | −1.76 | 3.21 | ˜u |
| U22970 | 16-jun gene, | −190 | 24 | 43 | 2363 | 0.2 | 53.95 | ˜u |
| | interferon-inducible | | | | | | | |
| | peptide (6-16) | | | | | | | |
| X57351 | 1-8d gene from | 2469 | 2686 | 24 | 297 | 0.09 | 113.8 | ˜u |
| | interferon-inducible | | | | | | | |
| | gene family | | | | | | | |
| X02874 | 2-5A synthetase (1.6 | 57 | −4 | −116 | 486 | −1.85 | 23.3 | ˜u |
| | kb) | | | | | | | |
| J00105 | beta-2 microglobulin | 1668 | 2101 | 248 | 1453 | 0.26 | 4.86 | ˜u |
| | gene | | | | | | | |
| M94880 | mhc class i (hla- | 551 | 421 | 202 | 932 | −0.31 | 3.61 | ˜u |
| | a*8001) | | | | | | | |
| HG2917 | major | 353 | 322 | 147 | 813 | −0.1 | 4.53 | ˜u |
| | histocompatibility | | | | | | | |
| | complex, class i, e | | | | | | | |
| D49824 | hla-b null allele | 527 | 267 | 144 | 925 | −0.97 | 5.42 | ˜u |
| Cluster2 | | | | | | | | |
| M98343 | amplaxin (ems1) | −24 | 63 | 15 | 141 | 2.15 | 6.05 | uu |
| Cluster3 | | | | | | | | |
| M92642 | alpha-1 type xvi | 11 | 115 | −147 | −133 | 4.75 | 0 | u˜ |
| | collagen (col16a1) | | | | | | | |
| J03909 | gamma-interferon- | 42 | 142 | 18 | 57 | 2.38 | 1.85 | u˜ |
| | inducible protein (ip- | | | | | | | |
| | 30) | | | | | | | |
| U41515 | dss1 | −62 | 123 | 104 | 51 | 5.15 | −1.04 | u˜ |
| Cluster4 | | | | | | | | |
| L40387 | thyroid receptor | 104 | 5 | 0 | 85 | −4.2 | 3.25 | du |
| | interactor (trip14) | | | | | | | |
| U72882 | interferon-induced | 125 | 27 | −9 | 502 | −3.63 | 24.1 | du |
| | leucine zipper | | | | | | | |
| | protein (ifp35) | | | | | | | |
| U53830 | interferon regulatory | 103 | 5 | −31 | 237 | −4.15 | 10.85 | du |
| | factor 7a | | | | | | | |
| M97935 | transcription factor | 331 | 92 | −37 | 296 | −2.6 | 13.8 | du |
| | isgf-3 | | | | | | | |
| U01824 | glutamate/aspartate | 339 | 35 | −73 | 91 | −8.69 | 3.55 | du |
| | transporter | | | | | | | |
| Cluster 5 | | | | | | | | |
| J00212 | leukocyte interferon | 106 | 9 | 138 | 49 | −4.3 | −1.82 | d˜ |
| | (ifn-alpha) | | | | | | | |
| U52513 | rig-g, | 289 | 10 | 49 | 96 | −13.45 | 0.96 | d˜ |
| X90846 | mixed lineage kinase | 357 | 60 | 425 | 173 | −4.95 | −1.46 | d˜ |
| | 2 | | | | | | | |
| L42243 | ifnar2 gene | 62 | −6 | 46 | −4 | −2.1 | −1.3 | d˜ |
| | (interferon receptor) | | | | | | | |
| M79462 | pml-1 | 201 | 11 | 43 | 1 | −9.05 | −1.15 | d˜ |
| K01900 | lymphocyte | 156 | 7 | 55 | 85 | −6.8 | 0.55 | d˜ |
| | interferon alpha type | | | | | | | |
| | 201 | | | | | | | |
| Cluster 6 | | | | | | | | |
| M30818 | interferon-induced | 20 | 5 | 756 | 155 | 0 | −3.88 | ˜d |
| | cellular resistance | | | | | | | |
| | mediator protein | | | | | | | |
| X15949 | interferon regulatory | 0 | 13 | 67 | 11 | 0 | −2.35 | ˜d |
| | factor-2 (irf-2) | | | | | | | |
| Column 1 = GenBank ID; column 2 = gene description; column 3 = nAD in CNCM I-2545 cultured in the absence of IFN-α; column 4 = nAD in CNCM I-2545 cultured in the presence of IFN-α; column 5 = nAD in CNCM I-2546 cultured in the absence of IFN-α; column 6 = CNCM I-2546 cultured in the presence of IFN-α; column 7 = change factor (CF) in CNCM I-2545; column 8 = change factor in CNCM I-2546; column 9 = cluster's characteristics | | | | | | | | |
| # (˜ = no change; d = downregulated; u = upregulated). | | | | | | | | |

Detection of Novel IFN-α Inducible Genes

Clusters 1 and 2, reported in Table 3, include genes previously unknown as IFN-α inducible, whose expression can be upregulated upon melanoma cell treatment. Cluster 1 comprises genes only induced in sensitive cells, whereas cluster 2 refers to genes upregulated upon IFN-α treatment of melanoma cells regardless of their “phenotypic” sensitivity. Some of these genes obviously belong to melanocytic (melanoma differentiation antigen, mda-6) or neuroectodermic (e.g., neuroleukin or catechol o-methyltransferase) cell lineages, while others clearly inducible genes such as, for instance, those encoding plasma gelsolin or spermidine synthase escape an evident, similar, tissue specific classification. Cluster 2 in Table 3 includes novel genes inducible regardless of IFN-α responsiveness in both lines analyzed. A number of these genes (rheb, PP1, ATPase, ceramidase, eif3) are functionally related to intracellular signaling pathways.

In Table 3 below, CNCM I-2546 and CNCM I-2545 cell lines cultured for 48 hours in the absence or in the presence of 100 U/ml IFN-α were used to identify novel cytokine induced genes whose expression was not found to be modulated in fibrosarcoma cells. Cluster 1 includes genes inducible in the CNCM I-2546 sensitive but not in the CNCM I-2545 resistant cell line. Cluster 2 comprises genes inducible in both lines. Data are presented as average difference of signal intensity between match and mismatch probesets.

TABLE 3
Novel IFN-α inducible genes

| | | CNCM I-2545 | CNCM I-2545 | CNCM I-2546 | CNCM I-2546 | CNCM I-2545 | CNCM I-2546 | |
|---|---|---|---|---|---|---|---|---|
| | | (−) | (+) | (−) | (+) | CF | CF | Cluster |
| Cluster 1 | | | | | | | | |
| L37043 | casein kinase i epsilon | 218 | 221 | 0 | 441 | 0.01 | 21.05 | ˜u |
| D32050 | alanyl-trna synthetase | 1458 | 761 | 21 | 427 | −0.92 | 19.33 | ˜u |
| D28137 | bst-2 | 37 | 88 | 28 | 305 | 1.38 | 9.89 | ˜u |
| S81914 | iex-1 = radiation-inducible | −66 | 21 | 31 | 312 | 0.05 | 9.06 | ˜u |
| | immediate-early gene | | | | | | | |
| X95325 | dna binding protein a | 634 | 253 | 58 | 375 | −1.51 | 5.47 | ˜u |
| | variant | | | | | | | |
| U91316 | acyl-coa thioester hydrolase | 253 | 428 | 141 | 706 | 0.69 | 4.01 | ˜u |
| U47025 | fetal brain glycogen | 672 | 465 | 193 | 924 | −0.45 | 3.79 | ˜u |
| | phosphorylase b | | | | | | | |
| Z26491 | gene catechol o- | 238 | 181 | 73 | 337 | −0.31 | 3.62 | ˜u |
| | methyltransferase | | | | | | | |
| L13210 | mac-2 binding protein | 2555 | 2092 | 444 | 2016 | −0.22 | 3.54 | ˜u |
| K03515 | neuroleukin | 2998 | 2552 | 487 | 2126 | −0.17 | 3.37 | ˜u |
| U09579 | melanoma differentiation | −59 | −26 | 83 | 361 | 0 | 3.35 | ˜u |
| | associated (mda-6) | | | | | | | |
| U72206 | guanine nucleotide regula- | 238 | 162 | 113 | 459 | −0.47 | 3.06 | ˜u |
| | tory factor (lfp40) | | | | | | | |
| X76538 | mpv17 | 132 | 101 | 77 | 313 | −0.31 | 3.06 | ˜u |
| U18009 | chromosome 17q21 clone | 674 | 423 | 187 | 735 | −0.59 | 2.93 | ˜u |
| | lf113 | | | | | | | |
| U69126 | fuse binding protein 2 | 114 | 159 | 77 | 302 | 0.39 | 2.92 | ˜u |
| | (fbp2) | | | | | | | |
| U50327 | protein kinase c substrate | 401 | 236 | 80 | 312 | −0.7 | 2.9 | ˜u |
| | 80k-h gene (prkcsh) | | | | | | | |
| D21235 | hhr23a protein | 388 | 346 | 150 | 582 | −0.12 | 2.88 | ˜u |
| HG1612 | macmarcks | 588 | 505 | 401 | 1440 | −0.16 | 2.59 | ˜u |
| X04412 | plasma gelsolin | 220 | 220 | 107 | 372 | 0 | 2.48 | ˜u |
| U65579 | mitochondrial nadh | 630 | 621 | 110 | 377 | −0.01 | 2.43 | ˜u |
| | dehydrogenase-ubiquinone | | | | | | | |
| Y00264 | amyloid a4 precursor | 392 | 427 | 89 | 304 | 0.09 | 2.42 | ˜u |
| M31013 | nonmuscle myosin heavy | 377 | 263 | 132 | 441 | −0.43 | 2.34 | ˜u |
| | chain (nmhc) | | | | | | | |
| M34338 | spermidine synthase | 472 | 277 | 429 | 1413 | −0.7 | 2.29 | ˜u |
| D50914 | EST | 234 | 78 | 110 | 359 | −2 | 2.26 | ˜u |
| U18018 | ela enhancer binding | 11 | 23 | 107 | 340 | 0.15 | 2.18 | ˜u |
| | protein (ela-f) | | | | | | | |
| U61263 | acetolactate synthase | 410 | 301 | 150 | 464 | −0.36 | 2.09 | ˜u |
| | homolog | | | | | | | |
| U65932 | extracellular matrix | 2498 | 1066 | 300 | 907 | −1.34 | 2.02 | ˜u |
| | protein 1 (ecm1) | | | | | | | |
| Cluster 2 | | | | | | | | |
| D78132 | rheb gene, ras-related gtp | 88 | 253 | 119 | 307 | 1.88 | 1.58 | uu |
| | binding protein gene | | | | | | | |
| M65028 | heterogeneous nuclear | 154 | 353 | 153 | 326 | 1.29 | 1.13 | uu |
| | ribonucleoprotein a/b | | | | | | | |
| X70848 | protein phosphatase 1, | 24 | 226 | 122 | 370 | 8.42 | 2.03 | uu |
| | catalytic subunit | | | | | | | |
| J04182 | lysosomal membrane | 66 | 484 | 168 | 452 | 6.33 | 1.69 | uu |
| | glycoprotein-1 (lamp1) | | | | | | | |
| J04444 | cytochrome c-1 gene | 886 | 2268 | 309 | 1286 | 1.56 | 3.16 | uu |
| L07633 | interferon-gamma | 22 | 275 | 184 | 463 | 11.5 | 1.52 | uu |
| L35249 | vacuolar h + -atpase mr | −40 | 357 | 92 | 227 | 16.85 | 1.47 | uu |
| | 56,000 subunit (ho57) | | | | | | | |
| M60784 | ul snrnp-specific protein | 115 | 413 | 24 | 216 | 2.59 | 8 | uu |
| U70063 | human acid ceramidase | 115 | 400 | 86 | 221 | 2.48 | 1.57 | uu |
| U78525 | human eukaryotic transla- | 165 | 372 | 275 | 589 | 1.25 | 1.14 | uu |
| | tion initiation factor (eif3) | | | | | | | |
| Z47055 | farnesyl pyrophosphate | 394 | 797 | 263 | 529 | 1.02 | 1.01 | uu |
| | synthetase like | | | | | | | |
| Column 1 = GenBank ID; column 2 = gene description; column 3 = nAD in CNCM I-2545 cultured in the absence of IFN-α; column 4 = nAD in CNCM I-2545 cultured in the presence of IFN-α; column 5 = nAD in CNCM I-2546 cultured in the absence of IFN-α; column 6 = CNCM I-2546 cultured in the presence of IFN-α; column 7 = change factor (CF) in CNCM I-2545; column 8 = change factor in CNCM I-2546; column 9 = cluster's characteristics | | | | | | | | |
| # (˜ = no change; d = downregulated; u = upregulated). | | | | | | | | |

1. A method for screening patients to determine their ability to respond to a tumor treatment, said method comprising:
measuring the expression level of at least one of the genes predictive for said treatment in patient samples; and
comparing the result of measurement to the result obtained with a reference sample.

2. A method as in claim 1, wherein patients are patients suffering from tumor.

3. A method as in claim 2, wherein patients are patients suffering from melanoma cancer.

4. A method as in claim 3, wherein the tumor treatment includes IFN-α or one of its derivatives.

5. A method as in claim 4, wherein gene expression is measured directly by DNA analysis with a DNA probe specific to at least one of the genes predictive for said treatment or by determination of the level of mRNA transcription or by determination of the level of gene product.

6. A diagnostic test for carrying out the method claimed in 1, comprising
contacting a matrix with probes with a liquid phase containing antibodies or nucleic acid probes
detecting gene transcription or product of one of the genes predictive for the tumor treatment.

7. A diagnostic test as claimed in 6, wherein the matrix comprises nucleic acids and the liquid phase contains target nucleic acid probes.

8. A diagnostic test as claimed in 6, wherein the matrix comprises target protein probes and the liquid phase contains antibodies.

9. A diagnostic kit for the method as claimed in 1 comprising a container with a matrix with probes.

10. A diagnostic kit as claimed in 9 comprising a container with a matrix with nucleic acid probes.

11. A method for screening the availability of cells or tissues to be sensitive or resistant to tumor treatment, said method comprising the identification of gene expression profiles characteristic of said treatment.

12. A method as in claim 11, wherein the cells originate from cell lines.

13. A method as in claim 11, wherein the cells originate from tumor cell lines.

14. An immunological marker enabling the selection of cells responding to a tumor treatment characterized in that it is an antibody specific for product of one or more of the genes predictive for said treatment.

15. A method for determining in a patient sample originating from a tumor, the presence or absence of expression of a gene predicative for treatment of the tumor with IFN-α or a pegylated IFN-α conjugate, said method comprising:
(a) obtaining from a patient having a tumor, a sample containing cells originating from the tumor; and
(b) detecting in the patient sample the expression of the gene,
wherein the presence of expression of the gene is predicative of the patient having an ability to respond to the treatment of the tumor using IFN-α or a pegylated IFN-α conjugate.

16. A method as in claim 15, wherein the tumor is ovary cancer, prostate cancer, breast cancer, colon cancer, liver cancer, stomach cancer or lung cancer.

17. A method as in claim 16, wherein the patient sample is prepared from blood, urine, serum, lymph node, bone marrow, cell extracts or tissue extracts.

18. A method as in claim 17, wherein the sample contains melanoma cells.

19. A method as in claim 18, wherein the pegylated-IFN-α conjugate has the formula: 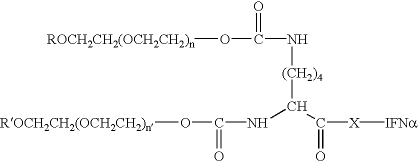
wherein R and R′ are independently lower alkyl; X is NH or O; n and n′ are integers having a sum of from 600 to 1500; and the average molecular weight of the polyethylene glycol in said conjugate is from about 26,000 Daltons to about 66,000 Daltons.

20. A method as in claim 19, wherein the pegylated-IFN-α conjugate has the formula: 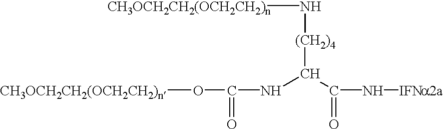
wherein n and n′ are independently 420 or 520.

21. A method as in claim 20, wherein the gene is at least one gene selected from the group consisting of S75415, M32053, X16665, and D00597.
